(19) United States
(12) Patent Application Publication
(10) Pub. No.: US 2025/0388927 A1
(43) Pub. Date: Dec. 25, 2025

(54) AAV GENE THERAPY FOR TREATING A NERVOUS SYSTEM DISEASE

(71) Applicant: SINEUGENE THERAPEUTICS CO., LTD., Bejing (CN)

(72) Inventors: Yichang JIA, Beijing (CN); Wei Guo, Beijing (CN); Lin PENG, Beijing (CN)

(21) Appl. No.: 18/879,098

(22) PCT Filed: Jun. 27, 2023
(86) PCT No.: PCT/CN2023/102706

§ 371 Date: Dec. 26, 2024

(30) Foreign Application Priority Data
Jun. 28, 2022 (WO) PCT/CN2022/101948

(51) Int. Cl. C12N 15/86; A61K 38/45; A61K 48/00; A61P 25/28; C12N 9/10

(52) U.S. Cl. CPC C12N 15/86; A61K 38/45; A61K 48/0058; A61P 25/28; C12N 9/104; C12N 2750/14143; C12N 2830/008

(57) ABSTRACT

Provided is a recombinant adeno-associated virus (rAAV) expression vector, comprising a gene encoding a TRIM72 protein or its variant or functional fragment thereof, wherein said recombinant AAV expression vector comprises a neuron-specific promoter. Further provided is a pharmaceutical composition comprising the rAAV expression vector and use thereof.

### BACKGROUND OF THE INVENTION

Amyotrophic lateral sclerosis (ALS), also known as Lou Gehrig's disease and motor neuron disease (MND), is a specific disease that causes the death of neurons which control voluntary muscles. Some also use the term “motor neuron disease” for a group of conditions of which ALS is the most common. ALS is characterized by stiff muscles, muscle twitching, and gradually worsening weakness due to muscles decreasing in size. This results in difficulty in speaking, swallowing, and eventually breathing. The cause is not known in 90% to 95% of case. About 5-10% of cases are inherited from a person's parents. About half of theses genetic cases are due to four specific genes, SOD1, TDP-43, FUS, and C9orf72. At present, there are only three clinical drugs for ALS treatment. One is riluzole, which was approved more than 20 years ago. The clinical trial results showed that riluzole can only prolong the survival of patients by several months. Edaravone, the second one, appears to be effective only in the early stages of the disease, slowing the progression of the disease by more than two months, but may actually accelerate the progression of the disease in intermediate and late stages. Patients in the AMX0035 treatment group showed only a slight statistically significant and clinically significant improvement, prolonging the patient's survival time with disease by 4.9 months. For drugs in clinical trials, there has been little breakthrough so far, only slowing disease progression by less than five months. Drug development for ALS has been difficult for decades. Now gene therapy is becoming the most promising treatment for neurodegenerative diseases including ALS. Gene therapy has made more molecules druggable. However, almost all of the current ALS gene therapy strategies are limited to targeting ALS patients with familial mutations, while there is no feasible solution for sporadic ALS patients. Oxidative stress is the common pathological process in all ALS patients, therefore, developing gene therapy drugs targeting oxidative stress is a potential feasible way to treat both familial and sporadic ALS.

TRIM72 is a Tripartite Motif (TRIM) family protein that consists of a Ring finger, a B-box motif, a coiled-coil region and a C-terminal PRYSPRY domain. It participates in sarcolemmal membrane repair process and is associated with insulin signaling pathway. It also takes part in cardioprotection against Ischemia/Reperfusion (IR) injury. Full-length TRIM72 work as a potential target for ALS through ubiquitinating mutant FUS protein has been reported. However, the effect of TRIM72 for protecting neurons needed to be further explored, and different kinds of TRIM72 protein/gene product needs to be further developed.

### SUMMARY OF THE INVENTION

The present disclosure provides recombinant adeno-associated virus (rAAV) expression vector, comprising a gene encoding a TRIM72 protein or its variant or functional fragment thereof. The rAAV expression vector has one or more of the following properties: (1) capable of highly express TRIM72 protein in vivo, for example, in neuron; (2) capable of protecting neurons effectively; (3) capable of reducing oxidative stress; (4) capable of treating, preventing and/or alleviating nervous system disease.

In one aspect, the present application provides a recombinant adeno-associated virus (rAAV) expression vector, comprising a gene encoding a TRIM72 protein or its variant or functional fragment thereof, wherein said recombinant AAV expression vector comprises a neuron-specific promoter.

In some embodiments, the neuron-specific promoter comprises a human derived promoter.

In some embodiments, the promoter is selected one or more from the group consisting of: an excitatory neuron-specific promoter, a brain neocortical and hippocampal excitatory neuron-specific promoter, a short neuron-specific promoter, a Dopaminergic neuron-specific promoter, a Glutaminergic neuron-specific promoter, a GABAergic neuron-specific promoter, a Cholinergic neuron-specific promoter, and a Serotoninergic neuron-specific promoter.

In some embodiments, the promoter is selected from a group selected from: human synapsin (hSyn), Calcium/calmodulin-dependent kinase IIa (CamKIIa), c-fos, methyl CpG-binding protein 2 (Mecp2), Neuron-specific enolase (NSE), somatostatin (SST), human vesicular GABA (Gamma-Aminobutyric Acid) transporter (hVGAT), choline acetyltransferase (ChAT), Serotonin transporter (SERT) and tyrosine hydroxylase (TH).

In some embodiments, the serotype of AAV vector is selected from AAV1, AAV2, AAV5, AAV6, AAV8, AAV9, AAVrh, AAVDJ, and AAVhull.

In some embodiments, the AAV is a single-strand AAV (ssAAV) or a self-complementary AAV (scAAV).

In some embodiments, the TRIM72 protein is a human TRIM72 protein.

In some embodiments, the TRIM72 protein comprises a full-length TRIM72 protein.

In some embodiments, the TRIM72 protein comprises a wild type TRIM72 protein.

In some embodiments, the human TRIM72 protein comprises an amino acid sequence as set forth in SEQ ID NO: 2.

In some embodiments, the TRIM72 protein comprises a TRIM72 truncated protein.

In some embodiments, the TRIM72 truncated protein comprises the PRYSPRY domain or its functional fragment of a TRIM72 protein.

In some embodiments, the PRYSPRY domain comprises amino acid sites of 278aa-470aa of the TRIM72 protein.

In some embodiments, the PRYSPRY domain comprises an amino acid sequence as set forth in SEQ ID NO: 6.

In some embodiments, the TRIM72 truncated protein further comprises the coiled-coil domain or its functional fragment of a TRIM72 protein.

In some embodiments, the TRIM72 truncated protein does not comprise the coiled-coil domain or its functional fragment of a TRIM72 protein.

In some embodiments, the coiled-coil domain comprises amino acid sites of 135aa-232aa of the TRIM72 protein.

In some embodiments, the coiled-coil domain comprises an amino acid sequence as set forth in SEQ ID NO: 5.

In some embodiments, the TRIM72 truncated protein further comprises the B-box domain or its functional fragment of a TRIM72 protein.

In some embodiments, the TRIM72 truncated protein does not comprise the B-box domain or its functional fragment of a TRIM72 protein.

In some embodiments, the B-box domain comprises amino acid sites of 86aa-117aa of the TRIM72 protein.

In some embodiments, the B-box domain comprises an amino acid sequence as set forth in SEQ ID NO: 4.

In some embodiments, the TRIM72 truncated protein further comprises the Ring-finger domain or its functional fragment of a TRIM72 protein.

In some embodiments, the TRIM72 truncated protein does not comprise the Ring-finger domain or its functional fragment of a TRIM72 protein.

In some embodiments, the Ring-finger domain comprises amino acid sites of 14aa-56aa of the TRIM72 protein.

In some embodiments, the Ring-finger domain comprises an amino acid sequence as set forth in SEQ ID NO: 3.

In some embodiments, the TRIM72 truncated protein comprises an amino acid sequence as set forth in any one of SEQ ID NO: 6, 7, 8, 9 and 11.

In some embodiments, the TRIM72 protein or its variant or functional fragment thereof comprises an amino acid mutation at position C14.

In some embodiments, the TRIM72 protein or its variant or functional fragment thereof comprises an amino acid mutation C14A.

In some embodiments, the TRIM72 protein or its variant or functional fragment thereof does not comprise an amino acid mutation at position C242.

In some embodiments, the rAAV expression vector is used for protect neurons by reducing oxidative stress.

In some embodiments, the rAAV expression vector is used for preventing or treating a nervous system disease.

In some embodiments, the rAAV expression vector is used for preventing or treating ALS, or Stroke.

In another aspect, the present application provides a host cell, comprising said rAAV expression vector.

In another aspect, the present application provides a pharmaceutical composition, comprises said rAAV expression vector or said host cell, and a pharmaceutically accepted adjuvant.

In another aspect, the present application provides a method for protecting neurons in a subject, comprising administering an effective amount of said rAAV expression vector, said host cell and/or said pharmaceutical composition to a subject in need thereof.

In another aspect, the present application provides a method for preventing and/or treating a nervous system disease, comprising administering an effective amount of said rAAV expression vector, said host cell and/or said pharmaceutical composition to a subject in need thereof.

In some embodiments, the nervous system disease comprising ALS, or Stroke.

In another aspect, the present application provides a use of said rAAV expression vector, said host cell and/or said pharmaceutical composition in manufacture of a medicament for preventing and/or treating a nervous system disease.

In some embodiments, the nervous system disease comprising ALS, or Stroke.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates different pAAV-ITR vectors containing TRIM72 or control genes. Each construct contains 5′ITR and 3′ITR for ssAAV or 5′ITR and 3′ITR-Δtrs for scAAV, an intron or not, neuron (hSyn1) or astrocyte (GfaABCID, a truncated GFAP promoter) specific promoter, mouse Trim72 (mTrim72) or human TRIM72 (hTRIM72) cDNA, SV40 polyadenylation signals (SV40 polyA) or bovine growth hormone polyadenylation signals (BGH polyA), followed by WHV's post-transcriptional regulatory elements (WPREs) or not.

FIG. 2 illustrates the administration procedure. Evaluation of neuropathological features in the SOD1-G93A transgenic mouse model show that impaired motor ability at about 80 days of age and continue until the mice reached the experimental endpoint. A significant decrease in body weight begins around 120 days of age and continue until the mice reach the experimental endpoint. It is expected that the mice will reach the experimental endpoint at 150 to 180 days of age. In the current experiment, we completed AAV injections in mice at 90 days of age. Mouse body weight was recorded from 90 days of age until they reached the experimental endpoint. Behavior tests in mice were performed from 108 days of age until 152 days of age.

FIG. 3 illustrates body weight of SOD1-G93A mice after ssAAV-TRIM72 treatment. All male mice were weighed every two days using a standard electronic scale. The change in body weight of mice was recorded after the injection of ssAAV (PHP.eB)-hSyn1-EGFP-TRIM72 or SSAAV (PHP.eB)-hSyn1-EGFP until the mice reached the experimental endpoint. Group size at start: EGFP: n=15; EGFP-TRIM72: n=15. *p<0.05 (Log rank).

FIG. 4 illustrates performance of locomotion behavior after ssAAV-TRIM72 treatment in SOD1-G93A mice. The total travel distance of mice was recorded in weekly open field tests starting at 108 days of age in male mice. The total travel distance of the mice showed significant differences in the open field test at 152 days of age. The values are presented as box plots with all data points. *p<0.05 (t-test), NS=no statistical significance.

FIG. 5 illustrates performance of rotarod behavior after ssAAV-TRIM72 treatment in SOD1-G93A mice. Five experiments were performed from day 112 to day 132 days of age in male mice. The stay time of mice in the experiments was recorded. In the last experiment, the mice injected with ssAAV-TRIM72 had a longer residence time and the difference was significant. The values are presented as box plots with all data points. *p<0.05 (t-test), NS=no statistical significance.

FIG. 6 illustrates survival curve of SOD1-G93A mice after ssAAV-TRIM72 treatment.

FIG. 7 EGFP-TRIM72 or EGFP expression in motor cortex and spinal cord after intravenous AAV delivery. (A) Representative fluorescent images of brain sections from SOD1-G93A mice injected with ssAAV (PHP.eB)-hSyn1-EGFP-TRIM72 or ssAAV (PHP.eB)-hSyn1-EGFP. (B) Representative fluorescent images of spinal cord sections from lumbar 4-5 position of SOD1-G93A mice treated with different AAVs. All the sections were stained for neurons (NeuN), transgene expression (GFP), and nucleus (DAPI), while additional ChAT staining was performed on spinal cord sections for motor neurons fluorescence.

FIGS. 8A-8B illustrates the relationship between ssAAV (PHP.eB)-EGFP or SSAAV (PHP.eB)-TRIM72-EGFP infection efficiency and animal survival days in SOD1-G93A mice.

FIG. 9 illustrates survival curve of TDP43-A315T mice after ssAAV-TRIM72 treatment and the relationship between ssAAV (PHP.eB)-EGFP or ssAAV (PHP.eB)-TRIM72-EGFP infection efficiency and animal survival days in TDP43-A315T mice.

FIG. 10 illustrates representative fluorescent images of astrocytes from wild type mice injected with ssAAV (PHP.eB)-GfaABCID-EGFP-TRIM72. Arrowheads indicated the astrocytes.

FIG. 11 illustrates no obvious exogenous TRIM72 expression in non-neuronal tissues. Flag-tagged TRIM72 expression across mouse cortex, spinal cord (S.C.), heart, kidney, liver, lung, muscle, and spleen. The tissues were from wild type C57BL/6 mice injected with ssAAV (PHP.eB)-hSyn1-EGFP-TRIM72 containing a Flag tag.

FIG. 12 illustrates performance of rotarod behavior after scAAV-hSyn1-TRIM72 treatment in SOD1-G93A mice. The stay time of mice in the experiments was recorded. At day 140, the mice injected with scAAV-hSyn1-TRIM72 had a longer stay time and the difference was significant. The values are presented as box plots with all data points. **p<0.01 (t-test).

FIGS. 13A-13B illustrates the therapeutic effects of TRIM72 in scAAV vectors on ALS animal models. scAAV9-hSyn1-TRIM72 treatment could significantly delay the disease progression indicated by 15% weight loss (A) and prolong median survival in SOD1-G93A mice (B). *p<0.05 (Log rank).

FIG. 14 illustrates the survival curve of TDP43-A315T mice after scAAV-TRIM72 treatment. scAAV-hSyn1-TRIM72 treatment group exhibited a longer median survival time while ssAAV-GfaABCID-TRIM72 treatment was less effective than the control.

FIGS. 15A-15B illustrates that loss-of-function of TRIM72 accelerates disease progression shown in FUS-R521C knockin mouse. (A) Loss-of-function of TRIM72 significantly shortened the life span of the FUS-R521C knockin mice. Mice, male, n=25 (+/+), 19 (C/C), 17 (C/C; −/−), and 18 (−/−). (B) Rotarod test was carried out at 4 months of age. Mice, male, n=10 (+/+), 10 (C/C), 12 (C/C; −/−), and 9 (−/−). The values are presented as mean±SEM. *p<0.05, **p<0.01, ***p<0.001, and ****p<0.0001 (ANOVA, SPSS). N.S., no statistical significance.

FIGS. 16A-16B illustrates TRIM72 was upregulated in neural but not non-neural tissue in mice with the indicated genotypes. (A) The total protein was visualized by Commassie blue. Cere., Cerebellum; Hippo., hippocampus. The quantification was shown in B and the values are presented as mean±SEM. ***p<0.001 (t-test), *p<0.05 (t-test), N.S., no statistical significance.

FIGS. 17A-17B illustrates the expression of TRIM72 in wildtype (+/+) and FUS-R521C (C/C) knockin mutant rats (A). GAPDH used as loading control. The quantification was shown in B and the values are presented as mean±SEM. ***p<0.001 (t-test), **p<0.01 (t-test), N.S., no statistical significance.

FIGS. 18A-18D illustrates TRIM72 is upregulated in ChAT-, Vglut1-, and Vgat-positive neurons in the FUS-R521C knockin mouse. ChAT-positive neurons: cholinergic neurons (lower motor neuron) in spinal cord, Vglut1-postive neurons: excitatory neurons in motor cortex. Vgat-positive neurons: inhibitory neurons in motor cortex. GFAP: marker for astrocyte. IBA1: marker for microglia. ChAT, GFAP and IBA1 were detected using immunostaining. TRIM72, Vglut1 and Vgat mRNAs were detected using RNAscope. The data summarizes were shown in B, C and D and the values are presented as mean±SEM. ***p<0.001 (t-test), N.S., no statistical significance.

FIG. 19 illustrates TRIM72 domain annotation and the key residues for TRIM72 functions were labeled.

FIG. 20 illustrates expression of Flag-tagged domain-disrupt TRIM72 fused with EGFP. GAPDH served as protein loading control. FL, full-length TRIM72.

FIG. 21illustrates domain-disrupt of TRIM72 on cell viability after stress challenge. The Coiled-coil domain-or PRYSPRY domain-disrupt not Ring domain-or B-box domain-disrupt abolished the protective effect of TRIM72. Values are presented as mean±SEM. ***p<0.001 (t-test), **p<0.01 (t-test), N.S., no statistical significance.

FIG. 22 illustrates different full-length and truncated TRIM72 or TRIM40 constructs in pAAV-ITR vectors.

FIG. 23 illustrates the cell viability under H₂O₂ stress with expression of full-length or different-truncated form of TRIM72 or full-length TRIM40 by scAAV9 infection. The values are presented as mean±SEM with all data points and t-test was performed to compare the datasets with control. **p<0.01 *p<0.05, N.S., no statistical significance.

FIG. 24 illustrates schematic diagram showing the timeline of treatments and behavior tests of middle cerebral artery occlusion/reperfusion (MCAO/R) model.

FIGS. 25A-B illustrates expression of full-length form of TRIM72 by scAAV9 infection promoted functional recovery after ischemia using neurological test and Adhesive-removal test in MCAO model. The values are presented as mean±SEM. **p<0.01 (t-test).

FIG. 26 illustrates the cell viability of N2a upon OGD treatment with expression of full-length form of TRIM72 by scAAV9 infection. The values are presented as mean±SEM. **p<0.01 (t-test), *p<0.05 (t-test).

FIG. 27 illustrates the expression of the TRIM72 mutants on cell viability after stress challenge. The C242A but not C14A mutation abolished the anti-stress effect of TRIM72. The values were generated from at least three independent experiments (n≥3). *p<0.05, **p<0.01, ***p<0.001 (ANOVA). N.S., no statistical significance

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

In the present application, the term “adeno-associated virus vector” generally refers to a nucleic acid derived from any AAV serotype, for example, AAV1, AAV2, AV3, AAV4, AAV5, AAV6, AAV7, AAV8, AAV9, AAV10, AAV11, AAV12 serotype, or any other virus or serotype that shares homologous in its capsid protein sequence to the capsid protein of an AAV serotype. The term “recombinant adeno-associated virus” or “rAAV” refers to an infectious, replication-defective virus composed of an AAV protein shell encapsulating a nucleic acid molecule of interest, which is flanked on one or both sides by AAV ITRs. As used herein, the reference to a particular AAV serotype means an AAV having at least one capsid protein of that AAV serotype. For example, the term “AAV9” refers to an AAV having at least one AAV serotype 9 capsid protein.

In the present application, the term “TRIM72 protein” can be used interchangeably with “MG53” protein, generally include a TRIM72 protein or its variant, functional fragment, analogue, homologue. The TRIM72 protein may contain a Ring finger, a B-box motif, a coiled-coil region and/or a C-terminal PRYSPRY domain. For example, the Ring-finger domain may comprise amino acid sites of 14aa-56aa of the TRIM72 protein or its functional fragment. For example, the B-box domain may comprise amino acid sites of 86aa-117aa of the TRIM72 protein or its functional fragment. For example, the coiled-coil domain may comprise amino acid sites of 135aa-232aa of the TRIM72 protein or its functional fragment. For example, the PRYSPRY domain may comprise amino acid sites of 278aa-470aa of the TRIM72 protein. The term may also include the TRIM72 protein derived from any known species which has a TRIM72 protein.

In the present application, the term “truncated protein” generally refers to a protein with one or more amino acid deletion compared with the full-length protein. For example, the truncated protein may contain the main functional fragment of the protein. For example, the truncated protein also includes but not limited to its variant, functional fragment, analogue, homologue.

In the present application, the “amino acid mutation Xn” refers to an amino acid mutation occurring in the amino acid residue X at position n of the amino acid sequence as set forth in SEQ ID NO: 2, wherein n is a positive integer, X is an abbreviation of any amino acid residue. For example, the “amino acid mutation C14” refers to the amino acid substitution occurring in the amino acid residue C corresponding to position 14 of the amino acid sequence as set forth in SEQ ID NO: 2.

The amino acid mutations of the present application can be non-conserved mutations. Said non-conserved mutations can comprise changing the amino acid residues in a target protein or polypeptide in a non-conserved manner, e.g., replacing an amino acid residue having a certain side chain size or a certain characteristic (e.g., hydrophilic) with an amino acid residue having a different side chain size or a different characteristic (e.g., hydrophobic).

Said amino acid substitutions can also be conserved substitutions. Said conserved substitutions can comprise changing the amino acid residues in a target protein or polypeptide in a conserved manner, e.g., replacing an amino acid residue having a certain side chain size or a certain characteristic (e.g., hydrophilic) with an amino acid residue having the same or similar side chain size or the same or similar characteristic (e.g., still hydrophilic). Such conserved substitutions generally would not produce a significant effect on the structure or the function of the produced protein. In the present application, the amino acid sequence variant which is a mutant of the fusion protein, its fragment, or its variant which undergoes one or more amino acid substitutions can comprise conserved amino acid substitutions that would not remarkably change the structure or function of the protein.

As an example, the mutual substitutions between amino acids in each of the following groups can be considered as conservative substitutions in the present application:

Group of amino acids with nonpolar side side(s): alanine, valine, leucine, isoleucine, proline, phenylalanine, tryptophan and methionine.

Group of uncharged amino acids with polar side chains: glycine, serine, threonine, cysteine, tyrosine, asparagine and glutamine.

Group of negatively charged amino acids with polar side chains: aspartic acid and glutamic acid.

Group of positively charged basic amino acids: lysine, arginine and histidine.

Group of amino acids with phenyl: phenylalanine, tryptophan and tyrosine.

In the present application, the term “vector” generally refers to a vector containing a recombinant polynucleotide, where the recombinant polynucleotide includes an expression control sequence efficiently linked to a nucleotide sequence to be expressed. The vector includes cis-acting elements sufficient for expression; other elements for expression may be provided by the host cell or may be provided in an in-vitro expression system. The vector may include all expression vectors known in the art that can be incorporated into the recombinant polynucleotide, including cosmid, plasmid (e.g., naked or encapsulated in liposomes) and viruses (e.g., lentiviruses, retroviruses, adenoviruses, and adeno-associated viruses).

In the present application, the term “encoding” generally refers to the inherent property of a particular sequence of nucleotides in a polynucleotide such as a gene, cDNA or mRNA to act as a template for the synthesis of other multimers and macromolecules in a biological process, said multimers and macromolecules having either a defined sequence of nucleotides (i.e., rRNA, tRNA and mRNA) or a defined sequence of amino acids and the biological properties arising therefrom. Thus, if transcription and translation of an mRNA corresponding to a gene produces a protein in a cell or other biological system, the gene encodes the protein. Both the coding strand whose nucleotide sequence is identical to the mRNA sequence and is usually provided in the sequence listing, and the non-coding strand used as a template for the transcription of a gene or cDNA may be referred to as the protein or other product encoding the gene or cDNA. In the present application, the term “coding element” generally refers to a nucleic acid (an RNA or DNA molecule) including a nucleotide sequence encoding a protein.

In the present application, the terms “host cell”, “cell”, and “host” are used interchangeably, and generally refer to a plasmid or vector that can include or have included the nucleic acid molecule of the present application, or can express individual cells, cell lines or cell cultures of the protein of the present application, its fragments or its variants. Said host cell can comprise the progeny of a single host cell. Due to natural, accidental or deliberate mutations, the progeny cells and the original parent cells can not necessarily be completely identical in morphology or genome, as long as they can express the protein of the present application or its fragments. Said host cell can be obtained by transfecting cells in vitro with the vector of the present application.

In the present application, the term “treat” generally refers to slowing or improving the progression, severity, and/or duration of a proliferative condition, or improving one or more symptoms (e.g., one or more distinguishable symptoms) of a proliferative condition as a result of the administration of one or more therapies.

In the present application, the term “subject” generally refers to any human or non-human animal. The term “non-human animal” can include all vertebrates, such as, mammals and non-mammals, e.g., non-human primates, goats, sheep, dogs, cows, chickens, amphibians, reptiles, etc.

In addition to particular proteins and nucleotides mentioned herein, the present application may also include their functional variants, derivatives, analogues, homologues and fragments thereof.

The term “functional variant” refers to a polypeptide having substantially the same amino acid sequence or encoded by substantially the same nucleotide sequence as the naturally occurring sequence and capable of having one or more activities of the naturally occurring sequence. In the context of the present application, the variant of any given sequence refers to a sequence in which a particular sequence of residues (either amino acid or nucleotide residues) has been modified so that the polypeptide or polynucleotide remains substantially at least one endogenous function. The variant sequences can be obtained through the addition, deletion, substitution, modification, replacement and/or variation of at least one amino acid residue and/or nucleotide residue present in a naturally occurring protein and/or polynucleotide, as long as the original functional activity is maintained. In the present application, the term “derivative” generally refers to a polypeptide or polynucleotide of the present application including any substitution, variation, modification, replacement, deletion and/or addition from/to one (or more) amino acid residues of the sequence, provided that the resulting polypeptide or polynucleotide substantially maintains at least one of its endogenous functions.

In the present application, the term “analogue” generally, with respect to a polypeptide or polynucleotide, includes any mimetic of the polypeptide or polynucleotide, that is, a chemical compound having at least one endogenous function of the polypeptide or polynucleotide that the mimetic mimics. In general, amino acids can be substituted, for example, at least 1 (e.g., 1, 2, 3, 4, 5, 6, 7, 8, 9, 10 or 20 or above) amino acids can be substituted, provided that the modified sequence substantially maintains the required activity or capability. Amino acid substitution may include the use of non-naturally occurring analogues. The protein or polypeptide used in the present application may also have deletion, insertion or substitution of amino acid residues, where the amino acid residues undergo silent changes and result in functionally equivalent proteins. Intentional amino acid substitutions can be made based on the similarity of the polarity, charge, solubility, hydrophobicity, hydrophilicity, and/or the amphoteric properties of the residues, as long as the endogenous function is retained. For example, negatively charged amino acids include aspartic acid and glutamic acid; positively charged amino acids include lysine and arginine; and amino acids containing uncharged polar head-groups with a similar hydrophilic value include asparagine, glutamine, serine, threonine and tyrosine.

In the present application, the term “homologue” generally refers to an amino acid sequence or a nucleotide sequence having a certain homology with a wild-type amino acid sequence and a wild-type nucleotide sequence. The term “homology” may be equivalent to the “identity” of sequences. Homologous sequences may include amino acid sequences that are at least 80%, 85%, 90%, 99.1%, 99.2%, 99.3%, 99.4%, 99.5%, 99.6%, 99.7%, 99.8% or 99.9% the same as the subject sequence. In general, homologues will contain the same active sites as the subject amino acid sequence, and the like. Homology may be considered on the basis of similarity (i.e., amino acid residues having similar chemical properties/functions), or homology can be expressed in terms of the sequence identity. In the present application, a sequence having a percentage identity in either of the SEQ ID NOs of the mentioned amino acid sequence or nucleotide sequence refers to a sequence having the percentage identity over the whole length of the mentioned SEQ ID NOs. In order to determine the sequence identity, alignment of sequences can be performed by a variety of ways known to those skilled in the art, for example, by using BLAST, BLAST-2, ALIGN, NEEDLE or Megalign (DNASTAR) software, etc. The persons skilled in the art are able to determine the suitable parameters suitable for alignment, including any algorithms required to achieve an optimal alignment in the full-length sequence being compared.

In the present application, the term “about” generally refers to varying in a range of 0.5%-10% above or below a specified value, for example, varying in a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below a specified value.

In the present application, the term “comprising” usually means including, containing, having or encompassing. In some cases, it also refers to the meaning of “being” or “consisting of”.

In the present application, the term “does not comprise” generally refers to the exclusion of the possibility of a certain behavior, structure or structure. For example, “A does not comprise B” generally means to exclude the possibility of B occurring in A.

Recombinant Adeno-Associated Virus (rAAV) Expression Vector

In one aspect, the present application provides a recombinant adeno-associated virus (rAAV) expression vector, comprising a gene encoding a TRIM72 protein or its variant or functional fragment thereof, wherein said recombinant AAV expression vector comprises a neuron-specific promoter.

In the present application, the rAAV may comprise an AAV genome or a derivative thereof, and/or an AAV capsid protein or a derivative thereof. In the present application, the rAAV may be a chimeric AAV, a shuffled AAV, or a capsid-modified AAV. In the present application, the AAV genome or AAV capsid protein may be from any one of AAV1, AAV2, AAV3, AAV4, AAV5, AAV6, AAV7, AAV8, AAV9, AAV10, AAV11, AAV12, AAVrh, AAVDJ, and AAVhull. In the present application, the rAAV may be a hybrid AAV (e.g., AAV-DJ, AAV-DJ/8, or AAV-DJ/9). In the present application, the rAAV may be developed through directed evolution and/or rational design (e.g., AAV 7m8 or AAV-PHP.B). In the present application, the rAAV may comprise one or more capsid mutations (e.g., Y-F, K-R, T-A, S-A and/or T-V mutations, (e.g., AAV2 with one or more capsid mutations among Y444F, Y500F, Y730F, Y252F, Y272F, Y700F, Y704F and T491V, or the corresponding mutation for a different AAV serotype, (e.g., AAV2/8 (Y733F), AAV2 (Y444F+Y500F+Y730F) and AAV2 (quadY-F+T-V))). In the present application, the serotype of the rAAV can be selected from the group consisting of AAV1, AAV2, AAV3, AAV4, AAV5, AAV6, AAV7, AAV8, AAV9, AAV10, AAV11, AAV12, Anc80, rh10 and ShH10. In the present application, the rAAV vector can be selected from the group consisting of AAV2/5, AAV2/8, AAV2/8 (Y733F), AAV2 (Y444F+Y500F+Y730F), AAV2/1, AAV2/4, AAV2/9, AAV2/6, AAV2/7, AAV1, AAV2, AAV4, AAV5, AAV6, AAV7, AAV8, AAV9, AAV12, Anc80, AAV 7m8, AAV-DJ, ShH10, AAV-PHP.B or a hybrid, a derivative or variant thereof. In the present application, the rAAV vector can be a single-stranded AAV vector or a self-complementary AAV (scAAV) vector.

In some embodiments, the serotype of AAV vector can be AAV9.

In the present application, an AAV packaging system can be used for delivery of a gene encoding TRIM72 protein or its variant or functional fragment thereof. For example, the AAV packaging system can be a triple-plasmid system. For example, the AAV packaging system may comprise a pAAV-ITR containing gene encoding TRIM72 protein or its variant or functional fragment thereof, a serotype vector pRepCapX and a helper vector pADHelper

In the present application, the neuron specific promoter can comprise a human derived promoter.

In the present application, the promoter can comprise a general neuron-specific promoter. In the present application, the promoter can comprise an excitatory neuron-specific promoter. In the present application, the promoter can comprise a brain neocortical and hippocampal excitatory neuron-specific promoter. In the present application, the promoter can comprise an inhibitory neuron-specific promoter. In the present application, the promoter can comprise a short neuron-specific promoter. In the present application, the promoter can comprise a Dopaminergic neuron-specific promoter. In the present application, the promoter can comprise a Glutaminergic neuron-specific promoter. In the present application, the promoter can comprise a GABAergic neuron-specific promoter. In the present application, the promoter can comprise a Cholinergic neuron-specific promoter. In the present application, the promoter can comprise a Serotoninergic neuron-specific promoter.

In the present application, the neuron specific promoter can be selected from a group selected from: human synapsin (hSyn), Calcium/calmodulin-dependent kinase IIa (CamKIIa), c-fos, methyl CpG-binding protein 2 (Mecp2), Neuron-specific enolase (NSE), somatostatin (SST), human vesicular GABA (Gamma-Aminobutyric Acid) transporter (hVGAT), choline acetyltransferase (ChAT), Serotonin transporter (SERT) and tyrosine hydroxylase (TH). For example, the promoter can be Syn1 promoter. For example, the Syn1 promoter may comprise a nucleotide sequence as set forth in SEQ ID NO: 12 or its variant thereof.

In the present application, the rAAV expression vector comprises a gene encoding a TRIM72 protein or its variant or functional fragment thereof.

In the present application, the TRIM72 protein can comprise a full-length TRIM72 protein. In the present application, the TRIM72 protein can comprise a wild type TRIM72 protein. In the present application, the human TRIM72 protein comprises an amino acid sequence as set forth in SEQ ID NO: 2.

In the present application, the TRIM72 protein can comprise a TRIM72 truncated protein.

In the present application, the TRIM72 truncated protein comprises the PRYSPRY domain or its functional fragment. For example, the TRIM72 truncated protein may comprises amino acid sites of 278aa-470aa of the TRIM72 protein. For example, the TRIM72 truncated protein may comprise an amino acid sequence as set for in SEQ ID NO: 6.

In the present application, the TRIM72 truncated protein may further comprise other domain of TRIM72 protein.

For example, the TRIM72 truncated protein may comprise the PRYSPRY domain and coiled-coil domain. For example, the TRIM 72 truncated protein may comprise a deletion of B-box domain and Ring-finger domain of TRIM72 protein. For example, the TRIM72 protein may comprise an amino acid sequence as set forth in SEQ ID NO: 11.

For example, the TRIM72 truncated protein may comprise the PRYSPRY domain and B-box domain. For example, the TRIM72 truncated protein may comprise a deletion of coiled-coil domain and Ring-finger domain of TRIM72 protein.

For example, the TRIM72 truncated protein may comprise the PRYSPRY domain and the Ring-finger domain. For example, the TRIM72 truncated protein may comprise a deletion of B-box domain and coiled-coil domain of TRIM72 protein.

For example, the TRIM72 truncated protein may comprise the PRYSPRY domain, the coiled-coil domain and the Ring-finger domain. For example, the TRIM72 truncated protein may comprise a deletion of B-box domain of TRIM72 protein.

For example, the TRIM72 truncated protein may comprise the PRYSPRY domain, the coiled-coil domain and the B-box domain. For example, the TRIM72 truncated protein may comprise a deletion of Ring-finger domain of TRIM72 protein.

For example, the TRIM72 truncated protein may comprise the PRYSPRY domain, the Ring-finger domain and the B-box domain. For example, the TRIM72 truncated protein may comprise a deletion of coiled-coil domain of TRIM72 protein.

In the present application, the TRIM72 protein or its fragments may comprise its variants. For example, the TRIM72 protein may comprise one or more amino acid mutations compared with the correspondence wild type sequence.

In the present application, the TRIM72 protein may comprise an amino acid mutation at position C14. For example, the amino acid mutation may be C14A.

According to applicant's research, amino acid cysteine at position 242 is critical for oligomer formation of TRIM72 protein. Substitution of amino acid C242 may block the protection function of neurons. Therefore, amino acid substitution at position C242 (for example, C242A) may not be included in the TRIM72 protein in the present application.

In the present application, the rAAV expression vector may be used for protect neurons by reducing oxidative stress.

In the present application, the rAAV expression vector may be used for preventing or treating a nervous system disease. For example, the present application may be used for preventing and/or treating ALS.

### Host Cell, Pharmaceutical Composition

In another aspect, the present application provides a cell (e.g., a host cell), which can comprise the rAAV expression vector of the present application.

In some embodiments, the host cell is a bacteria cell, an E. Coli cell, a plant cell, an insect cell, or a mammalian cell. In some embodiments, the cell is a somatic cell or a stem cell. In some embodiments, the rAAV expression vector is delivered to a host cell such that the Cas encoded by the nucleic acid molecule is expressed in the cell.

In another aspect, the present application provides a pharmaceutical composition, comprising said rAAV expression vector, or said host cell, and a pharmaceutically accepted adjuvant.

In some embodiments, pharmaceutically acceptable adjuvant can comprise buffers, antioxidants, preservatives, low molecular weight polypeptides, proteins, hydrophilic polymers, amino acids, sugars, chelating agents, counter-ions, metal complexes, and/or nonionic surfactants etc.

In the present application, said pharmaceutical composition can be formulated with a pharmaceutically acceptable carrier or diluent and any other known adjuvants and excipients according to conventional technical means in the art, e.g., following the operations in Remington: The Science and Practice of Pharmacy, nineteenth edition, edited by Gennaro, Mack Publishing Co., Easton, PA, 1995.

In the present application, said composition can be formulated for oral administration, intravenous administration, intramuscular administration, in situ administration at the tumor site, inhalation, rectal administration, vaginal administration, transdermal administration or the medicine is administered via a subcutaneous depot.

In the present application, said pharmaceutical composition can be used to protect neurons. For example, the composition of the present application can inhibit or delay the development or progression of nervous system diseases (e.g., ALS, or Stroke), and/or can reduce and/or stabilize the disease status.

The pharmaceutical composition of the present application can comprise a therapeutically effective amount of said rAAV expression vector. Said therapeutically effective amount is a dose required to prevent and/or treat (at least partially treat) diseases (e.g., ALS, or Stroke) and/or any complications thereof in a subject with or at a risk of the diseases.

### Preparation, Method and Use

In another aspect, the present application provides a method for protecting neurons in a subject, comprising administering an effective amount of the rAAV expression vector, the host cell, and/or the pharmaceutical composition to a subject in need thereof.

In another aspect, the present application provides a method for preventing and/or treating a nervous system disease, comprising administering an effective amount of the rAAV expression vector, the host cell, and/or the pharmaceutical composition to a subject in need thereof.

In another aspect, the present application provides a use of the rAAV expression vector, the host cell, and/or the pharmaceutical composition in manufacture of a drug for preventing and/or treating a nervous system disease.

In another aspect, the present application provides the rAAV expression vector, the host cell, and/or the pharmaceutical composition, for use in preventing and/or treating a nervous system disease.

In the present application, the nervous system disease may comprise a neurodegenerative disease or other disease.

In the present application, the nervous system disease comprises ALS, or Stroke. For example, the Stroke can be Ischemic Stroke.

### Examples

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular (ly); i.p., intraperitoneal (ly); s.c., subcutaneous (ly); rpm, revolutions per minute; and the like.

### Materials and Methods

### Animals

The ALS mouse model SOD1-G93A with overexpression of ALS-causing mutation SOD1^{G93A} or hTDP43-A315T generated by using a cDNA encoding human TDP43 with an N-terminal Flag tag with A315T mutation were used in the current project, while the age-matched wild-type (WT) mice were used as control in each experiment. The animal facility at Tsinghua University has been fully accredited by the Association for the Assessment and Accreditation of Laboratory Animal Care International (AAALAC) since 2014. All animal protocols were approved by the Institutional Animal Care and Use Committee (IACUC) at Tsinghua University based on the Guide for the Care and Use of Laboratory Animals (Eighth Edition, NHR). All mice were housed in isolated ventilated cages (maximum six mice per cage) barrier facility at Tsinghua University. The mice were maintained on a 12/12 h light/dark cycle, 22-26° C. 40-70% humidity with sterile pellet food and water ad libitum. Cages were checked daily to ensure animal welfare. Body weight was assessed regularly to ensure no weight loss. Where animals were used for research, we followed the 3Rs (replacement, refinement or reduction) rules.

### Behavior Tests

For the open field behavior test, the single animal was placed in the center of an open field area (60×60 cm) and tracked with multiple parameters, including total distance, average speed, and distance travelled in the center region using the TopScan behavioral analysis system (CleverSys, USA) at a 10-min interval. Rotarod performance was measured by an automated system (Med Associates Inc.). In brief, the animal was placed on an accelerating spindle (5-40 rpm) for 5 min per trial and three consecutive trials per day. A 20-min break was set in between each trial. The fall time from the spindle was auto-calculated by the system when the mouse fell off the spindle within the 5-min interval. The stay time was calculated by subtraction of the fall time from the 5 min, and the mean value of the stay time from three consecutive trials per day was used for statistical analysis. According to the age, genotype, and treatments, the mice were allocated to different experimental groups. The behavior assays were performed randomly in different groups. Adhesive-removal test was used to evaluate the neurological dysfunction after stroke, which is a sensitive method to assess sensorimotor deficits in focal cerebral ischemic mice. Briefly, a training session was performed until the mice could take off the sticky dots on their paws within 12 s before surgical procedures. Animals were tested 3, 5 or 7 days after ischemia by an investigator who was blind to the experimental groups. Both the time taken to sense the stimulus and to remove the tape are measured.

### Assessment of Experiment Endpoints

Mice were weighed three times a week and assessed for weakness, as previously described. The mice reached the endpoint of the experiment when a mouse was unable to right itself within 30 s after being put on its back.

### Immunoblot

Total protein content in cell or tissues were homogenized in RIPA buffer (50 Mm Tris-HCl, pH 8.0, 150 mM NaCl, 0.25% sodium deoxycholate, 0.1% SDS, 1% NP-40, supplemented with complete protease inhibitor mixture; Bimake B15001) and then the lysate was incubated for 30 minutes on ice. After 12000 rpm centrifugation for 10 minutes, the supernatant was extracted and was incubated at 95° C. for 10 minutes after mixing with SDS loading buffer. Next, the proteins were separated by 10% of sodium dodecyl sulfate-polyacrylamide gel electrophoresis (SDS-PAGE) and transferred onto a polyvinylidene fluoride (PVDF) membrane. Afterward, the membranes were blocked using 5% nonfat milk for 1 hours at room temperature. Then incubated with diluted primary antibodies overnight at 4° C., including GAPDH (Ameribio) TUBULIN (Ameribio), TRIM72 antibody (a kindly gift from Dr. Jianjie Ma), TSG101 (Abcam), ITGAV (Abcam), H3 (Abcam). And then the membranes were incubated with HRP-conjugated secondary antibodies at room temperature for 1 hour. Images were analyzed using the Fiji ImageJ to obtain the integrated intensities.

### Exosome Purification

The protocol for purifying exosomes from 100 ml supernatant of 293 FT cells included two steps of ultrafiltration and polyethylene glycol (PEG) precipitation. First, pour the collected supernatant into a centrifuge tube and centrifuge at 3000 xg for 20 minutes to remove cell debris. Then, filter the supernatant once with a 0.45 μm filter. After that, take a new Amicon ULTRA-15 ultrafiltration tube washed with PBS or autoclaved water. Then immediately add the supernatant to the ultrafiltration tube, and centrifuge at 3000 xg for 5-10 minutes. Discard the filtrate, continue to add the supernatant, and centrifuge at 3000 xg for 5-10 minutes until all the supernatant is introduced into the ultrafiltration tube. Then transfer the concentrate to a 50 mL centrifuge tube. Add isolation reagent to the concentrate and mix samples thoroughly by vortexing or pipetting. Finally, leave the samples at 2-8° C. overnight. The next day, samples were centrifuged at 10,000 xg for 1 hour at 4° C. Discard the supernatant, the exosomes were in the pellet.

### Middle Cerebral Artery Occlusion/Reperfusion (MCAO/R) Model

Male C57BL/6J mice weighing 25-28 g were used in this study. The mice were anesthetized with 1.2% Tribromoethanol. After the neck of mouse was shaved and disinfected with povidone-iodine solution, a midline incision was made to expose the carotid arteries, and then the common carotid artery (CCA) and external carotid artery (ECA) were ligated using 6-0 silk sutures to permanently occlude the blood flow. A silicone-coated nylon suture (MSMC23B100PK50, RWD) was introduced through an arteriotomy site in the CCA stump into the internal carotid artery (ICA) until feeling a mild resistance, indicating that it had reached the cranial base. Gently tightening the remaining loose monofilament suture around the CCA to prevent backflow leakage, the wound was then covered with cotton balls, and the mouse was transferred to a heating pad to maintain body temperature. After one hour, the silicone-coated nylon suture was withdrawn gently, and the monofilament suture was firmly ligated to avoid post-surgical bleeding. Finally, the wounds were closed with absorbable sutures.

### Oxygen-Glucose Deprivation (OGD)

During OGD, the medium was changed in a HEPES-buffered glucose-free medium that contained (in mmol/L) 154 NaCl, 5.6 KCl, 2.3 CaCl2, 1.0 MgCl2, 3.6 NaHCO3, 5HEPES, pH 7.4. OGD was induced by incubating N2a cells in this medium after scAAV9 infection (estimated multiplicity of infection (MOI): 10,000 vg/cell) was done for 24 hours, and then placing cells in an anaerobic chamber with an atmosphere of 95% N2 and 5% CO2 at 37° C. OGD was terminated after 2 h, 4 h or 6 h by replacing the glucose-free medium back to Neurobasal medium and further incubating the cultures in the normal cell culture incubator for 22 h or particularly indicated duration of re-oxygenation (Reperfusion, R) in individual experiments. Control cells were treated identically except that they were not exposed to OGD.

### Plasmids and Lentiviral Vectors

DNA fragments corresponding to full-length of TRIM72 were amplified from a mouse cDNA library by PCR and inserted into pCMV-N-3×Flag expression vector between SalI and XhoI sites using seamless Cloning kit (Beyotime) to generate Flag-tagged TRIM72. The series of Flag-tagged TRIM72 domain deletion: ΔRing domain (deletion of 14-69 domain); ΔB-box domain (deletion of 81-122aa); Δcoiled-coil domain (deletion of 135-232aa); ΔPRYSPRY domain (deletion of 278-470aa) construct was generated from the full-length of TRIM72 expression vector.

For TRIM72 stable expression cell line construction, DNA fragments of the series of Flag-tagged TRIM72 mutants or domain deletion or single domain were amplified from the corresponding TRIM72 expression vector by PCR and inserted into pLJM1-EGFP lentiviral vector between BsrG I and EcoR I sites to generate the series of Flag-tagged TRIM72 mutants or domain deletion or single domain fused with EGFP in lentiviral vector.

Lentivirus expressing Flag-tagged TRIM72 construct was generated from the corresponding EGFP fused with Flag-tagged TRIM72 vector.

### Cell Culture, Lentiviral Packaging and Lentiviral Infection

HEK293FT cells were maintained in DMEM (Invitrogen) with 10% fetal bovine serum (Gemini) in cell incubator (37° C., 5% CO2). For lentiviral packing, HEK293FT cells were seeded in growth medium for three 10-cm culture dish. When reaching around 90% confluence, cells were co-transfected with VSVG (10 μg), pxPAX2 (15 μg) and pLJM1-EGFP lentiviral vector or pLentiCRISPRv2 (Addgene) or pLenticas9-Blast (Addgene) (20 μg) using PEI (Sigma) following manufacturer's instructions and changed medium with fresh growth medium 5-6 h after transfection. The medium was harvested 72 hours after transfection and centrifugated at 20,000 rpm, 4° C. for 2 hours. After centrifugation, the lentivirus was enriched in the pellet. The lentivirus was resuspended using 100 μl DPBS and stored in −80° C.

HEK293FT cells or Hela cells were infected with indicated lentivirus. After 3 days of infection, the infected cells were selected with 2 μg/ml puromycin or 10 μg/ml blasticidin according to the plasmid containing resistance for at least a week. The puromycin-or blasticidin-selected cells were applied for further analysis.

### Cell Viability Assay

Cell viability was assessed using CCK-8. Cells were seeded in a 96-well plate at a density of1.5×10³ cells per well for Arsenite treatment or 7×10³cells per well for H₂O₂ treatment. Arsenite (Sigma) was added into each well at concentration of 0.125 mM, 0.25 mM or 0.5 mM, and washed the cells after 2 hours incubation at 37° C. For H₂O₂-treatment experiment, scAAV9 infection (estimated multiplicity of infection (MOI): 10,000 vg/cell) was done for 24 hours at 17 hours after cell seeding. Then, H₂O₂ was added into each well at concentration of 300 μM, and washed the cells after 1 hours incubation at 37° C. After cells have been processed by Arsenite or H₂O₂, a total of 10 μL of CCK-8 solution (Yeasen) was added to each well. After another 2 hours of incubation at 37° C., the optical density (OD) value of each well was measured using a microplate reader with an excitation wavelength of 450 nm. The cell viability of 293 FT was calculated. The experiment was repeated at least three times to obtain the mean value.

### Example 1 AAV Packaging and Construction of AAV Vectors

AAV packaging system is a commonly used triple-plasmid system. By simultaneously transfecting the three plasmids into mammalian cells (e.g. HEK293), all components required for AAV packaging can be expressed and assembled into virus particles in this cell. Here, we used a modified triple-plasmid system from PackGene (Guangzhou PackGene Biotech Co.,Ltd). This system consists of three plasmids: pAAV-ITR containing target gene, serotype vector pRepCapX and helper vector pADHelper. The target vector pAAV-ITR contains eukaryotic promoters and other components required for high levels of gene expression in mammalian gentral cells when foreign sequences are cloned into polyclonal sites (MCS). The vector also contains AAV reverse terminal repeat sequences (ITRs) that guide virus replication and packaging. PRepCapX contains AAV rep and CAP genes that encode replication proteins and viral capsid proteins. Stabilization of rep and CAP gene expression levels is a key step in obtaining desired high titer viral products. pADHelper contains a collection of adenovirus genes VA, E2A, and E4 that are essential for cell production of high-titer viruses.

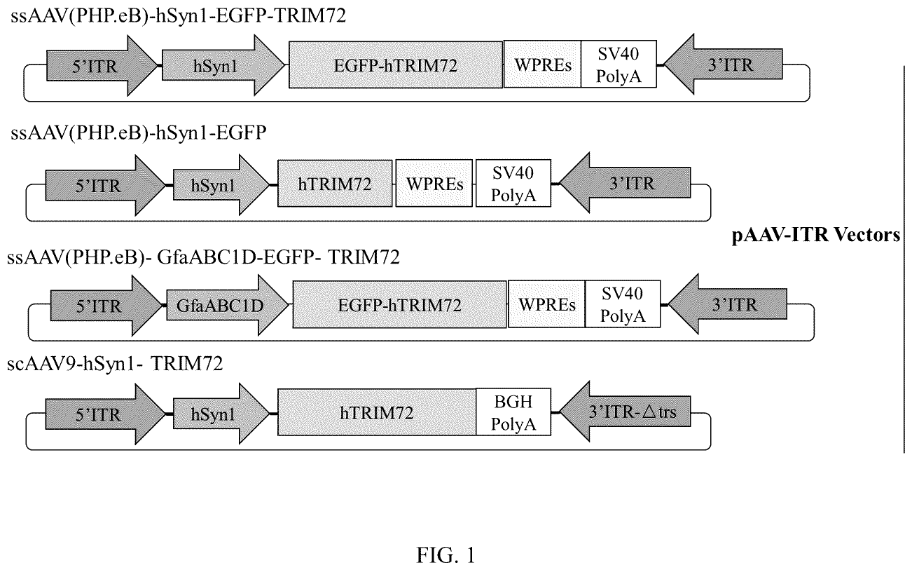

To construct pAAV-ITR plasmids for TRIM72 expression, we used both Self-complementary AAV (scAAV) and single-stranded (ss) AAV vectors. Compared with ssAAV9 vectors, scAAV9 vectors are 10- to 100-fold more efficient in transfection and expression, but can only package 2.2 kb foreign DNA. Moreover, scAAV persists as a stable episome in non-dividing cells with studies reporting stable transgene expression for years. FIG. 1 showed different pAAV-ITR vectors containing TRIM72 (SEQ ID NO: 2 or control genes. Human Synapsin1 (hSyn1) promoter is a neuron specific promoter. GfaABCID promoter, a truncated GFAP promoter, is an astrocyte specific promoter. Each construct contains 5′ITR and 3′ITR for ssAAV or 5′ITR and 3′ITR-Δtrs for scAAV, neuron (hSyn1) or astrocyte (GfaABCID) specific promoter, mouse TRIM72 (mTRIM72) or human TRIM72 (hTRIM72) cDNA, SV40 polyadenylation signals (SV40 polyA) or bovine growth hormone polyadenylation signals (BGH polyA), followed by WHV's post-transcriptional regulatory elements (WPREs) or not.

To deliver sufficient amount of AAV vectors to neural system and to provide sufficient levels of gene expression, the following (but not limited to) administration routes could be used: intracerebral administration, intrathecal administration, intravenous administration, aerosol administration, and intranasal, intramuscular, subcutaneous, intradermal, rectal, and other parental routes of administration. Routes of administration may be combined, if desired.

### Example 2 Therapeutic Effects of TRIM72 in ssAAV Vectors on ALS Animal Models

To examine the therapeutic effect of TRIM72 in AAV vectors on ALS, a classic ALS animal model SOD1-G93A transgenic mouse (The Jackson Lab: Stock #002726) and hTDP43-A315T transgenic mouse (The Jackson Lab: Stock #010700) were used here. Mutated SOD1 is an important genetic factor leading to the pathogenesis of ALS. SOD1-G93A transgenic mouse, which expresses high copy number of the mutated SOD1, develops adult-onset degeneration of spinal motor neurons and progressive motor defects, resulting in paralysis and death. TDP43-A315T transgenic mice express a mutant human TAR DNA binding protein cDNA harboring an amino acid substitution associated with familial ALS. Hemizygous mice develop a progressive and fatal neurodegenerative disease reminiscent of both ALS and frontotemporal lobar degeneration with ubiquitin aggregates.

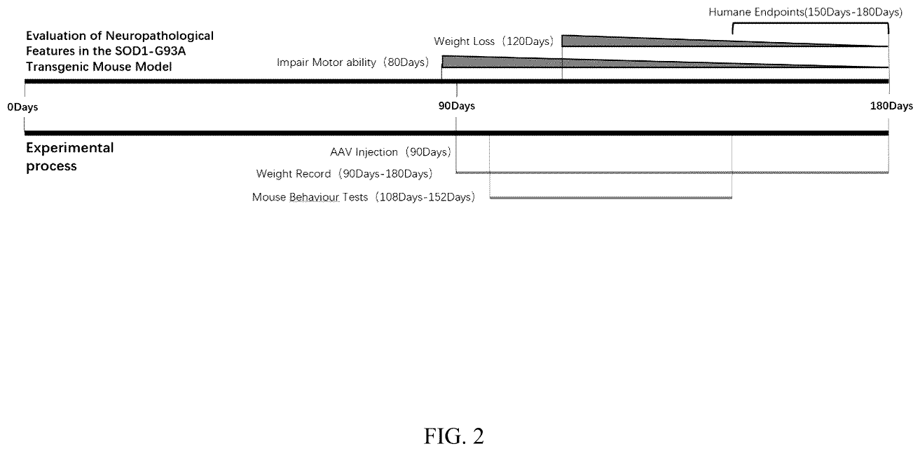

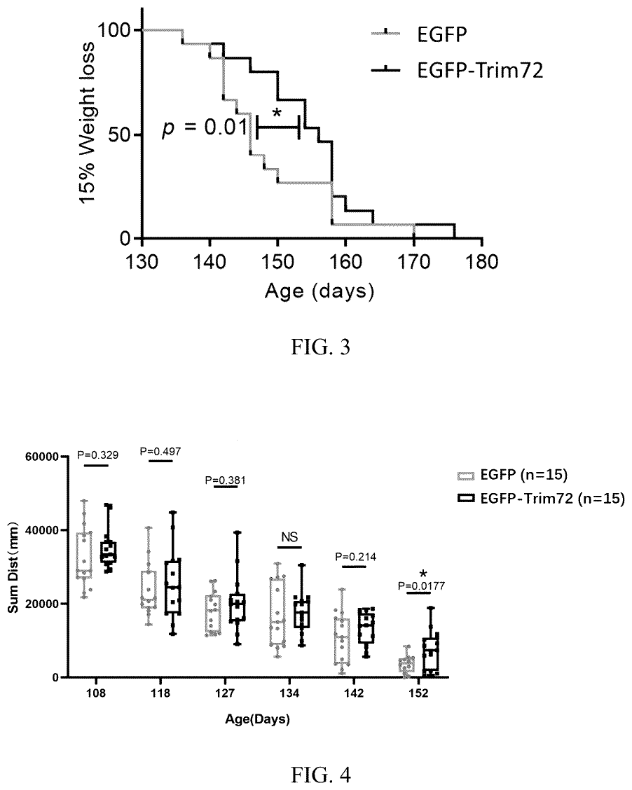

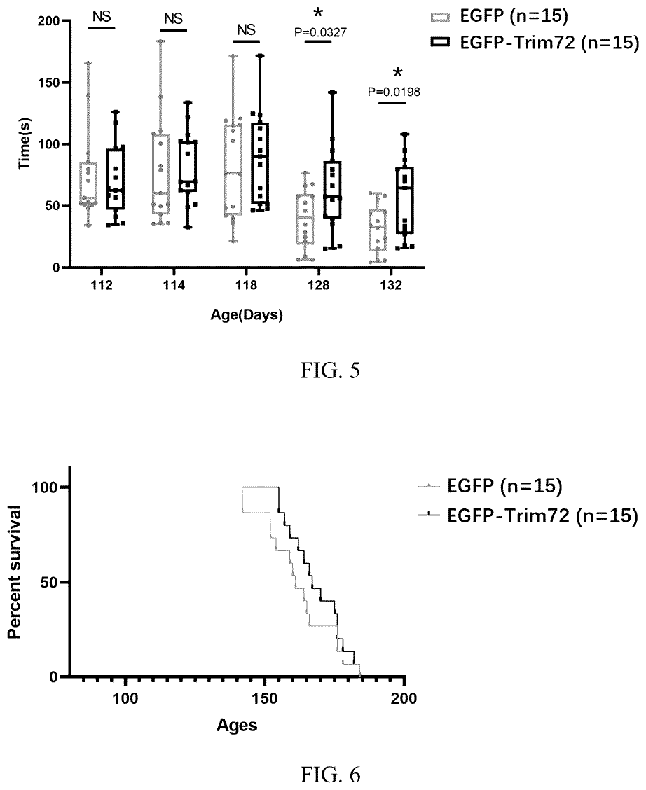

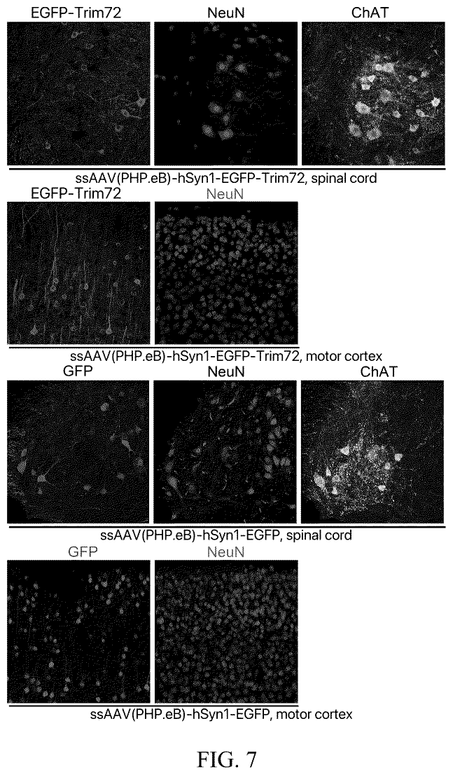

In the first experiment, ssAAV (PHP.eB)-EGFP-TRIM72 was used as the experiment group, and AAV (PHP.eB)-EGFP was served as the control. AAV vectors at a dose of 10¹¹ vg/mouse were injected into SOD1-G93A mice or at a dose of 3×10¹¹ vg/mouse were injected into TDP43-A315T mice respectively through retro-orbital intravenous injection according to the administration AAV (PHP.eB) procedure as shown in FIG. 2. The SOD1-G93A model mice showed a significant trend of weight loss at 130 days of age and continued until the end of the experiment. However, weight loss was relatively small in the EGFP-TRIM72 group compared to the AAV (PHP.eB)-EGFP group during disease progression. This indicated that AAV-TRIM72 had a moderate but significant protective effect on body weight loss due to disease progression in male mice (FIG. 3). Furtherly, to determine the effect of AAV-TRIM72 on the motor capability of SOD1-G93A, open field test for locomotion and rotarod test were performed during the treatment of AAV therapy. As the disease progressed, the total locomotor distance gradually decreased due to progressive death of motor neurons. It is suggested that AAV-TRIM72 has a partial protective effect on the reduced locomotor performance of SOD1-G93A mice (FIG. 4). Moreover, the treatment of AAV-TRIM72 significantly preserved motor skills as assessed by rotarod performance test, where there is a remarkable difference in latency to fall in mice at the age of 120 days and older (FIG. 5). Finally, the survival data showed that AAV-TRIM72 only had a mild effect on the survival of mice (FIG. 6). Furthermore, the survival time of TDP43-A315T mice was significantly prolonged after TRIM72 administration by ssAAV. The median survival in SSAAV (PHP.eB)-hSyn1-EGFP-TRIM72 treated group is about 133 days while in ssAAV (PHP.eB)-EGFP-treated group is about 117 days. It is suggested that AAV-TRIM72 has a protective effect on the lifespan extensions of TDP43-A315T mice (FIG. 9A).

To examine the expression of EGFP-TRIM72 in the target region including motor cortex and spinal cord, mouse tissues were sectioned and stained for GFP-tag, neuron, and motor neuron (FIG. 7). Infected neurons and motor neurons were observed in the motor cortex and spinal cord of SOD1-G93A transgenic mouse. The infection efficiency of ssAAV (PHP.eB)-hSyn1-EGFP-TRIM72 was calculated according to the ratio of infected neurons to total neurons. The efficiency was about 20.95% in the cortex and about 29.8% in the spinal cord. Further analysis also indicated the positive correlation between infection efficiency and days of animal survival in AAV (PHP.eb)-TRIM72-GFP-treated group but not in AAV (PHP.eb)-GFP-treated group in both SOD1-G93A and TDP43-A315T mice (FIG. 8 and FIG. 9B).

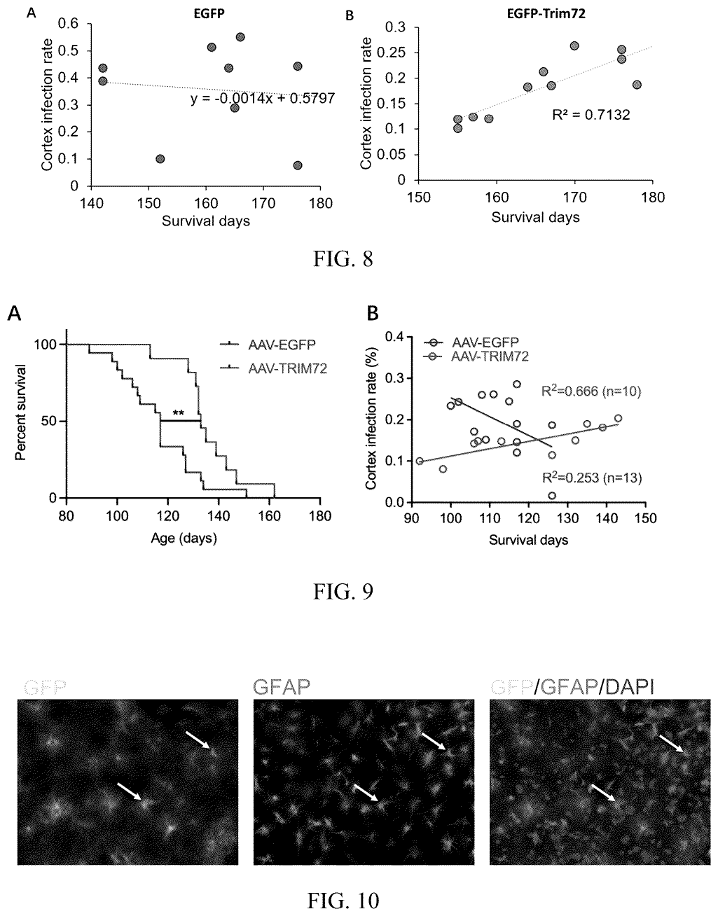

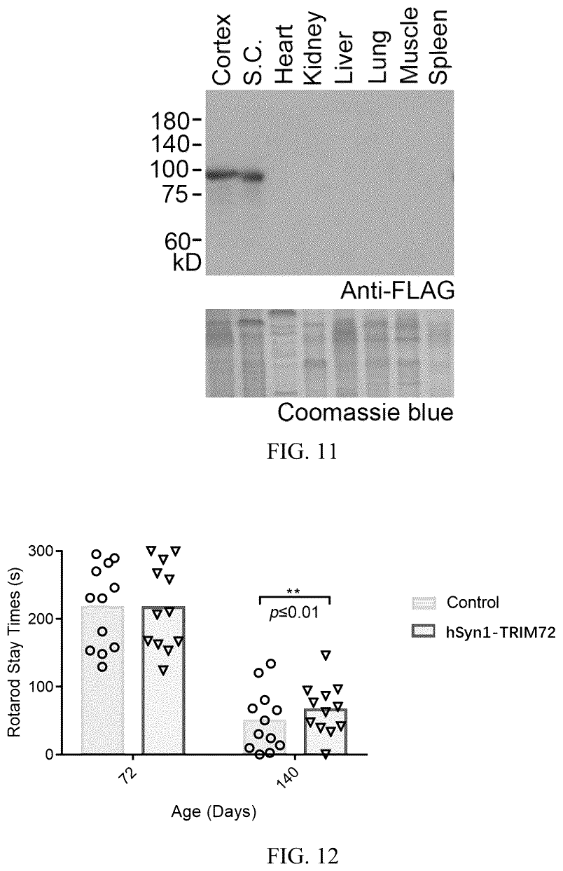

To characterize the expression of EGFP-TRIM72 in other non-neuronal tissues, the ssAAV (PHP.eB)-hSyn1-EGFP-TRIM72 construction were injected into wild type C57BL/6 mice at a dose of 10¹¹ vg/mouse, and tissues were collected for immunoblot after 10 days of expression. No obvious expression was detected in non-neuronal tissues such as heart, kidney, liver, lung, muscle, and spleen (FIG. 11). The expression of EGFP-Trim or GFP mainly distributed in cortex and spinal cord when hSyn1 promoter was used and mainly distributed in astrocyte when GfaABCID promoter was used (FIG. 10).

### Example 3 Therapeutic Effects of TRIM72 in scAAV Vectors on ALS Animal Models

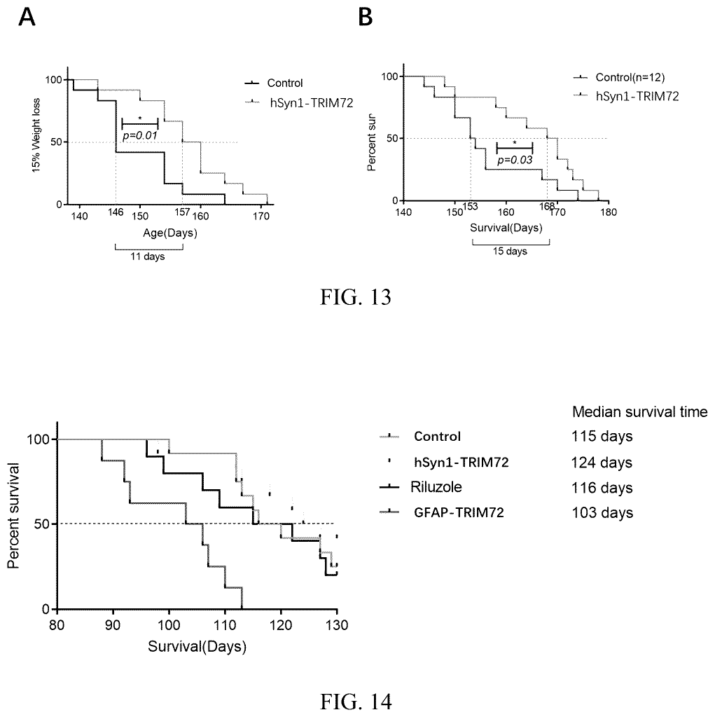

To achieve better therapeutic performance, a higher dose (2×10¹⁴ vg/kg body weight) of scAAV9-hSyn1-TRIM72 was used in the next experiments (the sequence of Syn1 promoter is as set forth in SEQ ID NO: 12). Vehicle was used as controls. Enhanced therapeutic effects were observed in the group of scAAV9-hSyn1-TRIM72 treatment. Improved motor functions were presented by rotarod behavior (FIG. 12). Moreover, scAAV9-hSyn1-TRIM72 treatment well delays the disease progression in SOD1-G93A mice indicated by 15% weight loss (FIG. 13A). Survival data also showed that scAAV9-hSyn1-TRIM72 at a dose of 2×10¹⁴ vg/kg body weight could significantly prolong median survival of SOD1-G93A mice (FIG. 13B). GfaABCID (SEQ ID NO: 13) is an astrocyte specific promoter. Another efficacy analysis was done in hTDP43-A315T mice, significant therapeutic effects were also observed in the group with scAAV (PHP.eB)-hSyn1-TRIM72 treatment at a dose of 8×10¹² vg/kg body weight but not in the group under ssAAV (PHP.eB)-GfaABCID-TRIM72 treatment at a dose of 10¹³ vg/kg body weight (FIG. 14). The median survival in scAAV (PHP.eB)-hSyn1-TRIM72 treated group is about 124 days while in control group is about 115 days. However, the ssAAV (PHP.eB)-GfaABCID-TRIM72 treatment group was less effective than the control group, the median survival is only 103 days. Riluzole was used as a reference, which is the same as the control group of median survival.

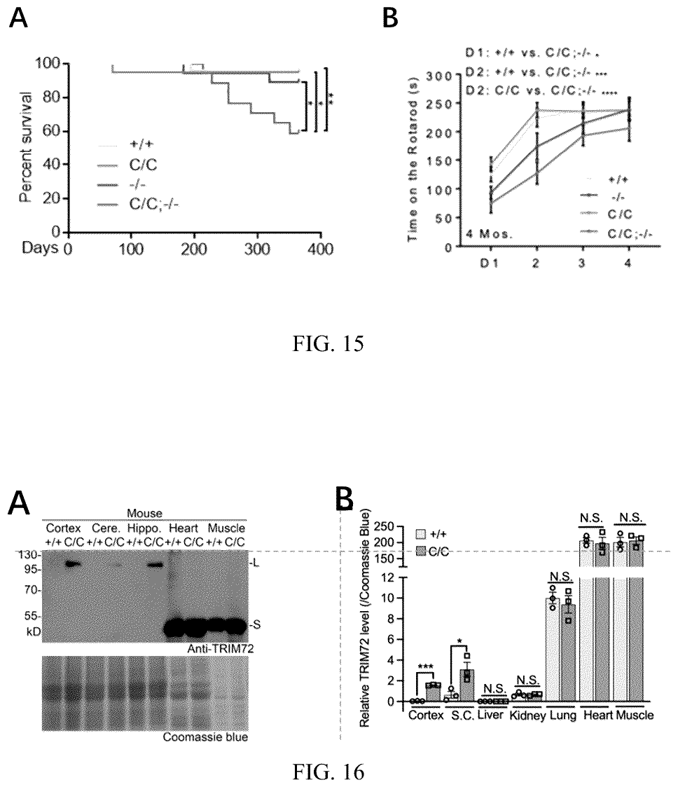

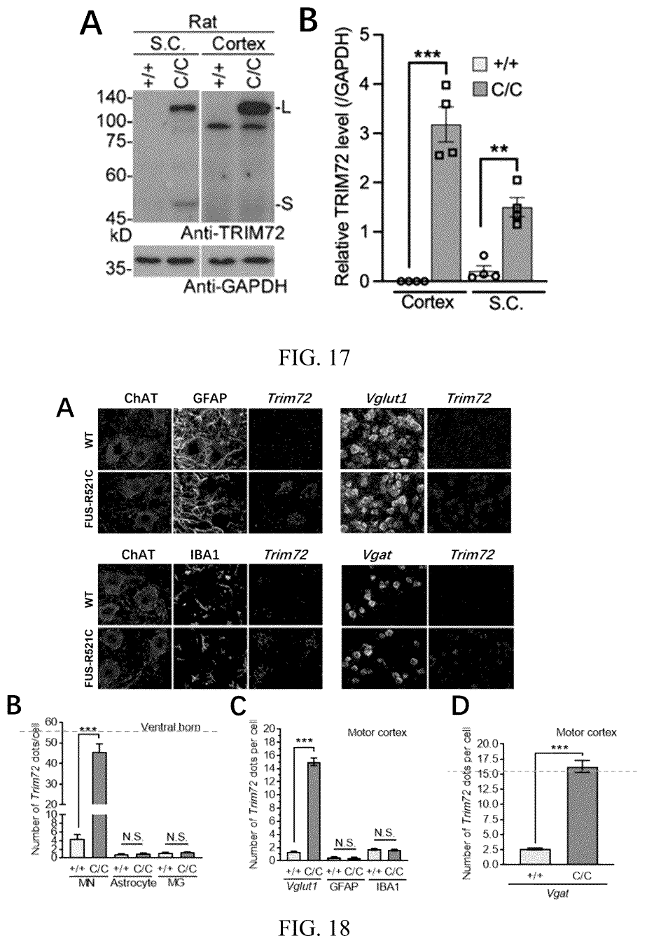

These results are also consistent with our previous findings. Specially, compared with the control group, FUS-R521C knock-in mice (C/C) did not exhibit severe ALS phenotype and had no significant difference in behavior indicated by rotarod test or survival curves (FIG. 15). Western analysis indicates that TRIM72 is overexpressed in the cortex and spinal cord in mice (C/C) and rats (C/C) (FIG. 16-17). Further analysis demonstrated that TRIM72 is specifically upregulated in neurons in FUS-R521C knock-in mice (C/C), but remains low-level in wildtype (+/+) (FIG. 18). Moreover, loss-of-function of Trim72 (−/−) could significantly shorten the stay time in rotarod test and the life span of the FUS-R521C knockin mice (FIG. 15). Thus, upregulation of TRIM72 in neurons is the critical element that FUS-R521C knock-in mice do not exhibit severe ALS phenotype. Therefore, specific expression of TRIM72 in neurons could achieve effective treatment for ALS and other neuron injury diseases.

### Example 4 Protective Effects of Truncated TRIM72 in scAAV Vectors in Culture Cells

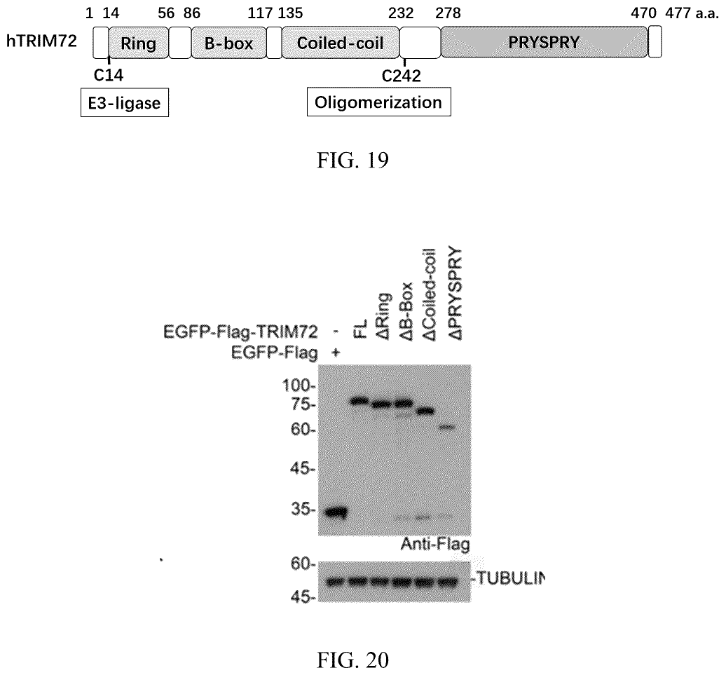

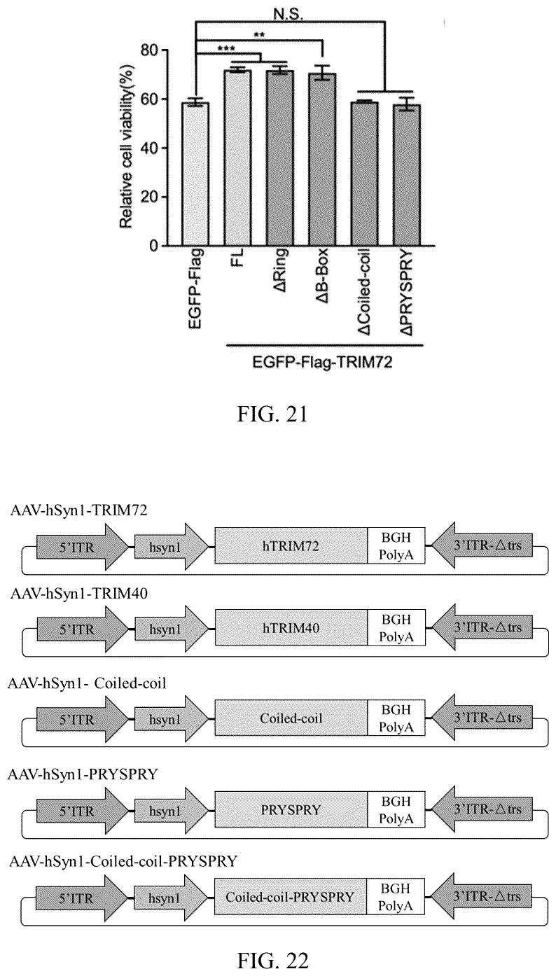

TRIM72 protein contains Ring finger motif, B-box domain, coiled-coil domain and PRYSPRY domain (FIG. 19). To investigate which domain of TRIM72 plays a role in protection from oxidative stress, full-length TRIM72 or domain-disrupted TRIM72 mutants were constructed and stably overexpressed in 293 FT cell line by lentiviral infection and puromycin-selection (FIG. 20). CCK-8 was used to measure the cell viability after Arsenite treatment. We found coiled-coil domain- and PRYSPRY domain-disrupted TRIM72 mutants abolished the protective effect of TRIM72, while Ring domain- or B-box domain-disrupted TRIM72 mutants reserved equal protective effect to wildtype (FIG. 21).

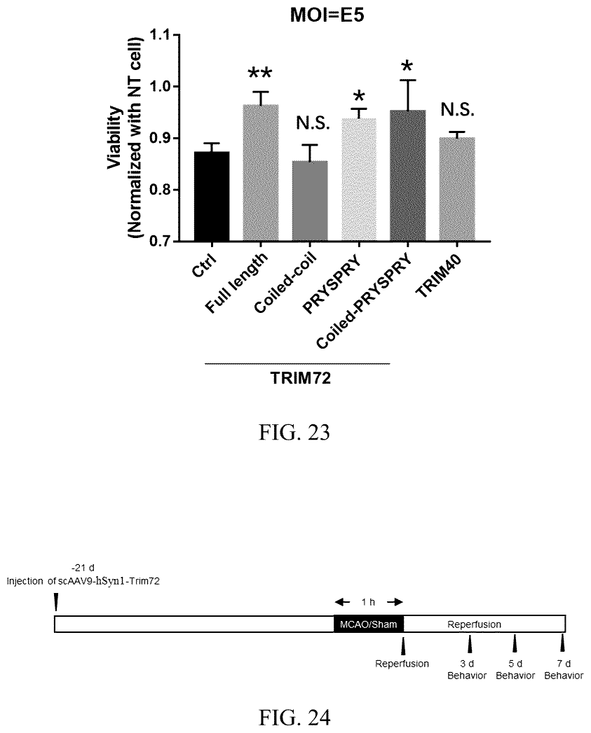

To further investigate which key domains of TRIM72 protein are necessary to protect neurons, different domain-disrupted TRIM72 were constructed and overexpressed in N2a cell line by scAAV9 infection at the same time (FIG. 22). CCK-8 was used to measure the cell viability after H₂O₂ treatment. Indeed, cell viability was decreased after H₂O₂ treatment (FIG. 23). It is noteworthy that overexpression of Coiled-PRYSPRY or the PRYSPRY single domain but not the coiled-coil single domain was sufficient to protect cells from oxidative stress, suggesting that either the Coiled-PRYSPRY truncation or the PRYSPRY single domain could elicit protective effect (FIG. 23).

### Example 5 scAAV-TRIM72 Prevent and Alleviate Brain Damage and Behavior Dysfunction in MACO/R Mice

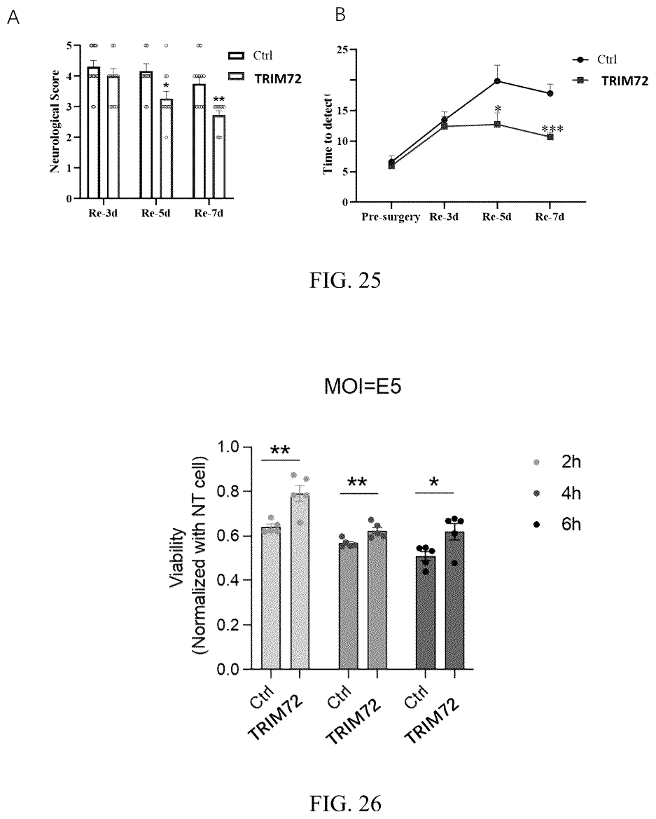

Here, an intraluminal middle cerebral artery occlusion/reperfusion (MCAO/R) model was used as a disease model for ischemic stroke. scAAV9-hSyn1-TRIM72 vectors at a dose of 4×10¹⁴ vg/kg body weight were injected into mice through retro-orbital intravenous injection according to the administration procedure as shown in FIG. 24. The quantification of TTC staining showed the size of the infarcted area of mice in scAAV-TRIM72 treated group was reduced compared with that in control group (data not shown). Moreover, it was demonstrated that scAAV-TRIM72 pre-treatment prevented and alleviated the behavioral deficits in motor and sensory activity task of the neurological test and adhesive-removal test at 5 days or 7 days after MCAO/R. scAAV-TRIM72 pre-treatment group had better behavioral scores (FIG. 25A), and can sense the presence of adhesive tape in shorter time after MCAO/R. (FIG. 25B).

### Example 6 scAAV-TRIM72 Prevents Cell Death Following OGD

The N2a cells experience increased oxidative stress and cell death when subjected to OGD conditioning. To investigate and confirm whether TRIM72 plays key role in protection after treatment of OGD. Full-length TRIM72 was overexpressed in N2a cell line by scAAV9 infection (estimated multiplicity of infection (MOI): 10,000 vg/cell) (FIG. 26), CCK-8 was used to measure the cell viability. Actually, cell viability was decreased after treatment of OGD, whereas overexpression of TRIM72 increased the cell viability (FIG. 26).

### Example 7. TRIM72 but not Other TRIM Proteins Protect Cells from Oxidative Stress

To investigate whether other TRIM proteins could also elicit protective effects from oxidative stress, TRIM40 were overexpressed in N2a cell line by scAAV9 infection (estimated multiplicity of infection (MOI): 10,000 vg/cell) (FIG. 22). CCK-8 was used to measure the cell viability after H₂O₂ treatment. Of note, treatment with TRIM72 rather than TRIM40 elicit protective effects from oxidative stress, which demonstrated that only TRIM72 but not other TRIMs could protect cells from oxidative stress (FIG. 23).

### Example 8. The Critical Sites of TRIM72 Protein which Protect Cells from Oxidative Stress

The series of Flag-tagged TRIM72 mutants: C14A (the cysteine at position 14 substituted by alanine); C242A (the cysteine at position 242 substituted by alanine) were generated from the wild-type TRIM72 construct by point mutation.

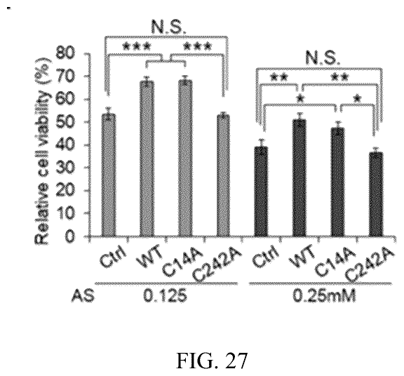

Previous study determined that TRIM72 senses changes in the oxidative environment and forms oligomer complex to complete membrane repair. A cystidine residue (C242) play a critical role in TRIM72 oligomer formation. In addition, the cystidine residue (C14) is critical for TRIM72 E3 ligase activity. Mutation of C242 into alanine (C242A) blocked TRIM72 protective effect, while its E3 ligase inactive mutant (C14A) reserved equal protective effect to wildtype (FIG. 27). It can be concluded that TRIM72 protects cells from oxidative stress and is mainly dependent on its oligomerization not E3 ligase activity.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A recombinant adeno-associated virus (rAAV) expression vector, comprising a gene encoding a TRIM72 protein or its variant or functional fragment thereof, wherein said recombinant AAV expression vector comprises a neuron-specific promoter.

2. The rAAV expression vector of claim 1, wherein said neuron-specific promoter comprises a human derived promoter.

3. The rAAV expression vector of claim 1, wherein said promoter is selected one or more from the group consisting of: an excitatory neuron-specific promoter, a brain neocortical and hippocampal excitatory neuron-specific promoter, a short neuron-specific promoter, a Dopaminergic neuron-specific promoter, a Glutaminergic neuron-specific promoter, a GABAergic neuron-specific promoter, a Cholinergic neuron-specific promoter and a Serotoninergic neuron-specific promoter.

4. The rAAV expression vector of claim 1, wherein said promoter is selected from a group selected from: human synapsin (hSyn), Calcium/calmodulin-dependent kinase IIa (CamKIIa), c-fos, methyl CpG-binding protein 2 (Mecp2), Neuron-specific enolase (NSE), somatostatin (SST), human vesicular GABA (Gamma-Aminobutyric Acid) transporter (hVGAT), choline acetyltransferase (ChAT), Serotonin transporter (SERT) and tyrosine hydroxylase (TH).

5. The rAAV expression vector of claim 1, wherein the serotype of AAV capsid is selected from AAV1, AAV2, AAV5, AAV6, AAV8, AAV9, AAVrh, AAVDJ, and AA Vhull.

6. (canceled)

7. (canceled)

8. The rAAV expression vector of claim 1, wherein said TRIM72 protein or its variant or functional fragment thereof comprises a full-length TRIM72 protein or a TRIM72 truncated protein.

9-11. (canceled)

12. The rAAV expression vector of claim 8, wherein said TRIM72 truncated protein at least comprises the PRYSPRY domain or its functional fragment of a TRIM72 protein.

13. (canceled)

14. The rAAV expression vector of claim 12, wherein said PRYSPRY domain comprises an amino acid sequence as set forth in SEQ ID NO: 6.

15-26. (canceled)

27. The rAAV expression vector of claim 1, wherein said TRIM72 truncated protein or its variant or functional fragment thereof comprises the amino acid sequence as set forth in any one of SEQ ID NO: 2, 6, 7, 8, 9 or 11.

28. The rAAV expression vector of claim 1, wherein said TRIM72 protein or its variant or functional fragment thereof comprises an amino acid mutation at position C14.

29. The rAAV expression vector of claim 1, wherein said TRIM72 protein or its variant or functional fragment thereof comprises an amino acid mutation C14A.

30. The rAAV expression vector of claim 1, wherein said TRIM72 protein or its variant or functional fragment thereof does not comprise an amino acid mutation at position C242.

31. The rAAV expression vector of claim 1, is used for protect neurons by reducing oxidative stress.

32. (canceled)

33. (canceled)

34. A host cell, comprising said rAAV expression vector of claim 1.

35. A pharmaceutical composition, comprises said rAAV expression vector of claim 1, and a pharmaceutically accepted adjuvant.

36. A method for protecting neurons in a subject, comprising administering an effective amount of said rAAV expression vector of claim 1 to a subject in need thereof.

37. A method for preventing and/or treating a nervous system disease, comprising administering an effective amount of said rAAV expression vector of claim 1 to a subject in need thereof.

38. The method of claim 37, wherein said nervous system disease comprising ALS, or Stroke.

39. (canceled)

40. (canceled)


SEQUENCE LISTING (13 sequences)

SEQ ID NO: 1 - protein, 477 aa, Mus musculus
MSAAPGLLRQELSCPLCLQLFDAPVTAECGHSFCRACLIRVAGEPAADGTVACPCCQAPTRPQALSTNLQLSRLVEGLAQVPQGHCEEHLDPLSIYCEQDRTLVCGVCASLGSHRGHRLLPAAEAQARLKTQLPQQKMQLQEACMRKEKTVAVLEHQLVEVEETVRQFRGAVGEQLGKMRMFLAALESSLDREAERVRGDAGVALRRELSSLNSYLEQLRQMEKVLEEVADKPQTEFLMKFCLVTSRLQKILSESPPPARLDIQLPVISDDFKFQVWKKMFRALMPALEELTFDPSSAHPSLVVSSSGRRVECSDQKAPPAGEDTRQFDKAVAVVAQQLLSQGEHYWEVEVGDKPRWALGVMAADASRRGRLHAVPSQGLWLLGLRDGKILEAHVEAKEPRALRTPERPPARIGLYLSFADGVLAFYDASNPDVLTPIFSFHERLPGPVYPIFDVCWHDKGKNAQPLLLVGPEQEQA

SEQ ID NO: 2 - protein, 477 aa, Homo sapiens
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 3 - protein, 43 aa, synthetic construct
  REGION 1..43: note = human TRIM72 Ring-finger domain 14-56 aa
CPLCLQLFDAPVTAECGHSFCRACLGRVAGEPAADGTVLCPCC

SEQ ID NO: 4 - protein, 32 aa, synthetic construct
  REGION 1..32: note = human TRIM72 B-box domain 86-117aa)
CEEHLDPLSIYCEQDRALVCGVCASLGSHRGH

SEQ ID NO: 5 - protein, 98 aa, synthetic construct
  REGION 1..98: note = human TRIM72 coiled-coil domain (135-232 aa)
QQKLQLQEACMRKEKSVAVLEHQLVEVEETVRQFRGAVGEQLGKMRVFLAALEGSLDREAERVRGEAGVALRRELGSLNSYLEQLRQMEKVLEEVADK

SEQ ID NO: 6 - protein, 193 aa, synthetic construct
  REGION 1..193: note = human TRIM72 PRYSPRY domain (278-470 aa)
RKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLV

SEQ ID NO: 7 - protein, 421 aa, synthetic construct
  REGION 1..421: note = TRIM72 Ring domain (deletion of 14-69aa)
MSAAPGLLHQELSQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 8 - protein, 435 aa, synthetic construct
  REGION 1..435: note = TRIM72 B-box domain (deletion of 81-122aa)
MSAAPGLLHQELSCPLCLQLFDAPVTAECGHSFCRACLGRVAGEPAADGTVLCPCCQAPTRPQALSTNLQLARLVEGLAQAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 9 - protein, 379 aa, synthetic construct
  REGION 1..379: note = TRIM72 coiled-coil domain (deletion of 135-232aa)
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 10 - protein, 284 aa, synthetic construct
  REGION 1..284: note = TRIM72 PRYSPRY domain (deletion of 278-470aa)
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWGPEGAEA

SEQ ID NO: 11 - protein, 336 aa, synthetic construct
  REGION 1..336: note = TRIM72 coiled-coil domain plus PRYSPRY domain (135-470aa)
QQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLV

SEQ ID NO: 12 - DNA, 470 bp, synthetic construct
  misc_feature 1..470: note = Human Synapsin1 (hSyn1) promotor
  source 1..470: mol_type = other DNA
tctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 13 - DNA, 681 bp, synthetic construct
  misc_feature 1..681: note = GfaABC1D (Truncated GFAP) promoter
  source 1..681: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc